(19) United States
(12) Patent Application Publication
(10) Pub. No.: US 2015/0079180 A1
(43) Pub. Date: Mar. 19, 2015

(54) NANOPARTICLE COMPOSITIONS OF DIMETHYL FUMARATE

(71) Applicant: XenoPort, Inc., Santa Clara, CA (US)

(72) Inventors: Sami Karaborni, Cupertino, CA (US); Chen Mao, Mountain View, CA (US); Garry T. Gwozdz, Jim Thorpe, PA (US)

(21) Appl. No.: 14/490,277

(22) Filed: Sep. 18, 2014

Related U.S. Application Data
(60) Provisional application No. 61/879,327, filed on Sep. 18, 2013.

(51) Int. Cl. A61K 31/225; A61K 47/38; A61K 47/20; A61K 9/14

(52) U.S. Cl. CPC A61K 31/225; A61K 9/14; A61K 47/38; A61K 47/20
USPC 424/489; 560/190; 514/547

(57) ABSTRACT

Disclosed herein are compositions of dimethyl fumarate exhibiting reduced gastrointestinal irritation and related side effects.

### CROSS-REFERENCE

This application claims the benefit under 35 U.S.C. §119(e) of U.S. Provisional Application Ser. No. 61/879,327, filed Sep. 18, 2013, and entitled “NANOPARTICLE COMPOSITIONS OF DIMETHYL FUMARATE,” the contents of which is incorporated by reference in its entirety.

### FIELD

Disclosed herein are novel compositions of dimethyl fumarate which achieve high therapeutic blood plasma concentrations of monomethyl fumarate in patients, while avoiding serious gastrointestinal irritation and related side-effects.

### BACKGROUND

Dimethyl fumarate refers to the dimethyl ester of fumaric acid. The compound has a molecular weight of 144.13 daltons and the following chemical structure:

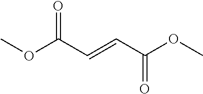

This compound is also known by the names Dimethyl (E)-butenedioate (IUPAC), trans-1,2-Ethylenedicarboxylic acid dimethyl ester and (E)-2-Butenedioic acid dimethyl ester. The compound is also referred to by the acronym DMF. DMF can be synthesized according to the methods described in Chinese Patent Publication CN 101318901A, the disclosures of which are incorporated herein by reference. The compound in crystalline form has a disclosed melting point of between 102° C. and 105° C. Dimethyl fumarate is rapidly metabolized in vivo to monomethyl fumarate (MMF), and hence DMF is considered to be a prodrug of MMF.

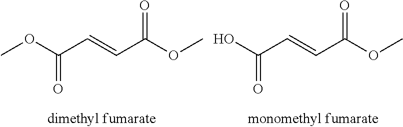

Fumaderm®, an enteric coated tablet containing a mixture of dimethyl fumarate and salts of monoethyl fumarate, was approved in Germany in 1994 for the treatment of psoriasis. Fumaderm® is sold as an enteric-coated oral tablet dosage form and is available in two different dosage strengths (Fumaderm® initial and Fumaderm®):

| | Fumaderm ® | Fumaderm ® |
|---|---|---|
| Fumarate Compound | Initial (mg) | (mg) |
| Dimethylfumarate | 30 | 120 |
| Ethyl hydrogen fumarate, calcium salt | 67 | 87 |
| Ethyl hydrogen fumarate, | 5 | 5 |
| magnesium salt | | |
| Ethyl hydrogen fumarate, zinc salt | 3 | 3 |

The two strengths are intended to be applied in an individually based dosing regimen starting with Fumaderm® initial in an escalating dose, and then after, e.g., three weeks of treatment, switching to Fumaderm®.

Another marketed composition is Fumaraat 120® containing 120 mg of dimethyl fumarate and 95 mg of calcium monoethyl fumarate (TioFarma, Oud-Beijerland, Netherlands). The pharmacokinetic profile of Fumaraat 120® in healthy subjects is described in Litjens et al., Br. J. Clin. Pharmacol., 2004, vol. 58:4, pp. 429-432. The results show that a single oral dose of Fumaraat 120® is followed by a rise in serum monomethyl fumarate concentration and only negligible concentrations of dimethyl fumarate and fumaric acid are observed.

Tecfidera™, formerly called BG-12, is a delayed release oral dosage form (i.e., a capsule containing enteric-coated minitablets) of dimethyl fumarate. Tecfidera™ (dimethyl fumarate) was approved in the USA in 2013, and is dosed two times per day at 480 mg/day for the treatment of multiple sclerosis. Details concerning the clinical testing of BG-12 are disclosed in Sheikh et al., Safety Tolerability and Pharmacokinetics of BG-12 Administered with and without Aspirin, Key Findings from a Randomized, Double-blind, Placebo-controlled Trial in Healthy Volunteers, Poster PO4.136 presented at the 64th Annual Meeting of the American Academy of Neurology, Apr. 21-28, 2012, New Orleans, La.; Dawson et al., Bioequivalence of BG-12 (Dimethyl Fumarate) Administered as a Single 240 mg Capsule and Two 120 mg Capsules: Findings from a Randomized, Two-period Crossover Study, Poster P913 presented at the 28th Congress of the European Committee for Treatment and Research in Multiple Sclerosis, Oct. 10-13, 2012, Lyon, France; and Woodworth et al., Pharmacokinetics of Oral BG-12 Alone Compared with BG-12 and Interferon β-1a or Glatiramer Acetate Administered Together, Studied in Health Volunteers, Poster PO4.207 presented at the 62nd Annual Meeting of the American Academy of Neurology, Apr. 10-17, 2010, Toronto, Ontario, Canada.

U.S. Pat. Nos. 6,277,882 and 6,355,676 disclose, respectively, the use of alkyl hydrogen fumarates and the use of certain fumaric acid monoalkyl ester salts for preparing microtablets for treating psoriasis, psoriatic arthritis, neurodermatitis and enteritis regionalis Crohn. U.S. Pat. No. 6,509,376 discloses the use of certain dialkyl fumarates for the preparation of pharmaceutical preparations for use in transplantation medicine or the therapy of autoimmune diseases in the form of microtablets or micropellets. U.S. Pat. No. 4,959,389 discloses compositions containing different salts of fumaric acid monoalkyl esters alone or in combination with a dialkyl fumarate. GB Patent No. 1,153,927 relates to medical compositions comprising dimethyl maleic anhydride, dimethyl maleate and/or dimethyl fumarate.

Dimethyl fumarate is highly irritating to the skin and mucosal membranes with the result that oral administration tends to cause serious digestive tract irritation with attendant nausea, vomiting, abdominal pain and diarrhea. For example, Fumaderm® dosing frequently causes irritation of the gastric and intestinal tissues, which in turn causes fullness, diarrhea, upper abdominal cramps, flatulence and/or nausea. Similarly, Tecfidera™ dosing frequently causes abdominal pain, diarrhea, nausea, vomiting and dyspepsia. Unfortunately, these gastrointestinal side effects limit the utility of dimethyl fumarate for treating diseases such as psoriasis and multiple sclerosis.

### SUMMARY

The present disclosure describes nanoparticle compositions of dimethyl fumarate. In some aspects, the present disclosure describes nanoparticle compositions comprising dimethyl fumarate, hydroxypropylmethyl cellulose and an anionic surfactant. In other aspects, the disclosure describes pharmaceutical compositions comprising the nanoparticles.

In certain embodiments the nanoparticles have a weight ratio of dimethyl fumarate to hydroxypropylmethyl cellulose in a range of about 1:1 to about 10:1.

In other embodiments, the nanoparticles have a weight ratio of dimethyl fumarate to anionic surfactant in a range of about 30:1 to about 300:1.

In other embodiments, the nanoparticles have a median diameter ranging from about 50 to about 400 nm.

In other embodiments, the hydroxypropylmethyl cellulose has a molecular weight in a range of about 2,000 to about 100,000 daltons.

Also disclosed are oral dosage forms containing a therapeutically effective amount of dimethyl fumarate-containing nanoparticles.

Also disclosed are methods of treating a disease in a patient in need of such treatment, comprising administering to the patient a therapeutically effective amount of the dimethyl fumarate-containing nanoparticles. In certain embodiments, the disease is one of multiple sclerosis and psoriasis.

### BRIEF DESCRIPTION OF THE DRAWINGS

FIG. 1 shows the particle size distribution of the nanoparticulate suspension of Example 1 after 1 hour of milling.

FIG. 2 shows the particle size distribution of the nanoparticulate suspension of Example 4 after 5 minutes of milling.

FIG. 3 shows the particle size distribution of the nanoparticulate suspension of Example 4 after 30 minutes of milling.

FIG. 4 shows the particle size distribution of the nanoparticulate suspension of Example 4 after 2.5 hours of milling.

FIG. 5 shows dissolution rates of a nanoparticulate dimethyl fumarate suspension compared with microparticulate dimethyl fumarate suspensions.

### DEFINITIONS

The term “Monomethyl fumarate” refers to the monomethyl ester of fumaric acid. The compound has the following chemical structure:

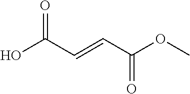

and has a molecular weight of 130.10 daltons. The compound is also commonly referred to as 2(E)-Butenedioic acid 1-methyl ester, (2E)-4-Methoxy-4-oxobut-2-enoic acid; Fumaric acid hydrogen 1-methyl ester; (2E)-2-Butenedioic acid 1-methyl ester; (E)-2-Butenedioic acid monomethyl ester; Monomethyl trans-ethylene-1,2-dicarboxylate; and methyl hydrogen fumarate. The compound is also referred to herein and elsewhere by the acronyms MMF and/or MHF.

The terms “nanoparticle” and “nanoparticulate” refer to particles having a median diameter of less than 1,000 nm or a median diameter ranging from about 10 to about 1,000 nanometers (nm). The particles can be in the form of powder, dry powder or in the form of an aqueous suspension, a hydrogel, an emulsion, a liposome, or a micelle.

“Patient” refers to a mammal, for example, a human.

“Pharmaceutically acceptable” refers to approved or approvable by a regulatory agency of the Federal government or a state government or listed in the U.S. Pharmacopoeia or other generally recognized pharmacopoeia for use in animals, and more particularly in humans.

The terms “pharmaceutically acceptable vehicle” and “pharmaceutically acceptable carrier” refer to a pharmaceutically acceptable diluent, a pharmaceutically acceptable adjuvant, a pharmaceutically acceptable excipient, or a combination of any of the foregoing, with which a composition provided by the present disclosure may be administered to a patient, which does not destroy the pharmacological activity thereof and which is non-toxic when administered in doses sufficient to provide a therapeutically effective amount of the composition.

“Subject” refers to either a human or a non-human, such as primates, mammals, and vertebrates.

“Systemic administration” and “systemically administering” shall each mean a route of administration of a compound into the circulatory system of a patient in a therapeutically effective amount. In some non-limiting embodiments, administration can take place via enteral administration (absorption of the medication through the gastrointestinal tract) or parenteral administration (generally injection, infusion, or implantation). These terms are in contrast with topical and other types of local administration where a therapeutically effective amount is not in the circulatory system.

“Treating” or “treatment” of any disease refers to reversing, alleviating, arresting, or ameliorating a disease or at least one of the clinical symptoms of a disease, reducing the risk of acquiring at least one of the clinical symptoms of a disease, inhibiting the progress of a disease or at least one of the clinical symptoms of the disease or reducing the risk of developing at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to protecting against or delaying the onset of at least one or more symptoms of a disease in a patient.

The term “therapeutically effective amount” refers to an amount sufficient to produce a desired therapeutic effect, for example, an amount that is sufficient to achieve a desired therapeutic effect. The actual amount required for treatment of any particular patient will depend upon a variety of factors including the disorder being treated and its severity; the specific pharmaceutical composition employed; the age, body weight, general health, sex and diet of the patient; the mode of administration; the time of administration; the route of administration; the rate of excretion; the duration of the treatment; any drugs used in combination or coincidental with the specific compound employed; the discretion of the prescribing physician; and other such factor(s) known to those skilled in the art. These factors are discussed in Goodman and Gilman's “The Pharmacological Basis of Therapeutics”, Tenth Edition, A. Gilman, J. Hardman and L. Limbird, eds., McGraw-Hill Press, p. 155-173, 2001.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

### DETAILED DESCRIPTION

Dimethyl fumarate (DMF) is a prodrug of monomethyl fumarate. Once administered, the compound is metabolized in vivo into an active metabolite, namely, monomethyl fumarate (MMF) which is also referred to herein as methyl hydrogen fumarate (MHF). The in vivo metabolism of dimethyl fumarate to monomethyl fumarate is illustrated below:

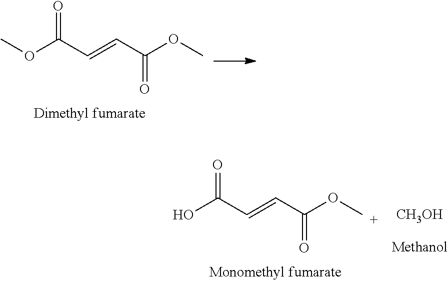

Dimethyl fumarate is known to have limitations on absorption at high doses. DMF absorption is also known to be highly variable. In addition, DMF is known to cause GI irritation. These issues can be due to the relatively low solubility of DMF and the relatively high dose that is required for effective therapy in MS and psoriasis patients. The present disclosure is directed towards overcoming these problems.

Thus, in some aspects, the present disclosure provides compositions comprising dimethyl fumarate nanoparticles.

In other aspects, the present disclosure provides pharmaceutical compositions comprising DMF nanoparticles.

In certain embodiments, the pharmaceutical compositions are in the form of a suspension, a tablet, a pill, a capsule, a sustained release formulation or a powder.

The nanoparticle compositions of the present disclosure have increased surface area compared to larger sized particles, e.g., particles in the μm size range. Increasing the surface area of drug particles increases dissolution rates and supersaturation concentrations in solution, thereby improving delivery efficiency and bioavailability for commonly used routes of administration such as oral administration. Thus, the nanoparticle compositions of the disclosure have, or are expected to have, advantageous pharmaceutical properties including improved solubility, improved absorption rates, improved bioavailability, reduced GI irritation and related side effects, and reduced residence time in the GI tract.

In certain embodiments, the nanoparticle compositions are in a liquid form or a solid form. The solid form can be for example a powder, a dry powder or a lyophilized powder.

In certain embodiments, the nanoparticle compositions are in the form of a suspension, a hydrogel, or an emulsion.

In some embodiments, the nanoparticle compositions are in the form of a suspension. In other embodiments, the nanoparticle compositions are in the form of an aqueous suspension.

In some embodiments, the nanoparticles have a median diameter less than 1,000 nanometers (nm). In other embodiments, the nanoparticles have a median diameter ranging from about 10 to about 1,000 nm. In other embodiments, the nanoparticles have a median diameter ranging from about 10 to about 500 nm. In other embodiments, the nanoparticles have a median diameter ranging from about 10 to about 400 nm. In other embodiments, the nanoparticles have a median diameter ranging from about 10 to about 300 nm. In other embodiments, the nanoparticles have a median diameter ranging from about 10 to about 200 nm. In other embodiments, the nanoparticles have a median diameter ranging from about 10 to about 150 nm. In other embodiments, the nanoparticles have a median diameter ranging from about 10 to about 100 nm.

In some embodiments, the nanoparticles have a median diameter ranging from about 50 to about 500 nm. In other embodiments, the nanoparticles have a median diameter ranging from about 50 to about 400 nm. In other embodiments, the nanoparticles have a median diameter ranging from about 100 to about 300 nm. In other embodiments, the nanoparticles have a median diameter ranging from about 100 to about 200 nm. In other embodiments, the nanoparticles have a median diameter of about 150 nm.

In certain embodiments, the nanoparticle compositions comprise one or more excipients. Examples of excipients that may be used in one or more of the nanoparticle compositions disclosed herein can be found in “Handbook of Pharmaceutical Excipients” Rowe et al., editors, 7^{th} edition, Pharmaceutical Press. London, 2012.

In certain embodiments, the nanoparticle compositions comprise one or more surfactants.

Examples of surfactants that may be used include polysorbates; sodium dodecyl sulfate (sodium lauryl sulfate), lauryl dimethyl amine oxide, docusate sodium, cetyl trimethyl ammonium bromide (CTAB), a polyethoxylated alcohol, a polyoxyethylene sorbitan, octoxynol, N,N-dimethyldodecylamine-N-oxide, hexadecyl trimethylammonium bromide, polyoxyl 10 lauryl ether, brij, a bile salt such as sodium deoxycholate or sodium cholate, a polyoxyl castor oil, nonylphenol ethoxylate, a cyclodextrin, lecithin, methylbenzethonium chloride, a carboxylate, a sulphonate, a petroleum sulphonate, an alkylbenzenesulphonate, a naphthalenesulphonate, an olefin sulphonate, a sulphate surfactant, an alkyl sulphate, a sulphated natural oil or fat, a sulphated ester, a sulphated alkanolamide, an alkylphenol, an alkylphenol that is optionally ethoxylated and/or sulphated, an ethoxylated aliphatic alcohol, polyoxyethylene, a carboxylic ester, a polyethylene glycol ester, an anhydrosorbitol ester or an ethoxylated derivative thereof, a glycol ester of a fatty acid, a carboxylic amide, a monoalkanolamine condensate, a polyoxyethylene fatty acid amide, a quaternary ammonium salt, an amine with amide linkages, a polyoxyethylene alkyl amine, a polyoxyethylene alicyclic amine, a N,N,N,N-tetrakis substituted ethylenediamine, a 2-alkyl-1-hydroxyethyl-2-imidazoline, N-coco-3-aminopropionic acid or a sodium salt thereof, N-tallow-3-iminodipropionate disodium salt, N-carboxymethyl-N-dimethyl-N-octadecenyl ammonium hydroxide, N-cocoamidoethyl-N-hydroxyethylglycine sodium salt, or combinations of any of the foregoing.

In certain embodiments, the nanoparticle compositions comprise one or more of a polymer, a copolymer, or an anionic surfactant.

In certain embodiments, the nanoparticle compositions comprise a monomer, a polymer, a hydrogel, an emulsion, a liposome, a micelle, a complexing ligand or a hydrotropic agent.

In some embodiments, the nanoparticle compositions comprise a polymer. The polymer may be any polymer capable of improving the solubility and stability of the nanoparticle compositions.

In some embodiments, the nanoparticle compositions comprise a polymer and the nanoparticles have a weight ratio of dimethyl fumarate to the polymer in a range of about 1:1 to about 10:1. In certain embodiments, the nanoparticles have a weight ratio of dimethyl fumarate to the polymer in a range of about 1:1 to about 8:1. In certain embodiments, the weight ratio is in a range of about 1:1 to 6:1. In certain embodiments, the weight ratio is in a range of about 1:1 to 4:1. In certain embodiments, the weight ratio is in a range of about 1:1 to 2:1. In particular embodiments, the nanoparticles have a weight ratio of dimethyl fumarate to the polymer that is about 5:1.

In some embodiments, the polymer is a cellulose based polymer.

Suitable examples of cellulose based polymers include, but are not limited to, cellulose derivatives such as hydroxypropylcellulose, hydroxymethylcellulose, hydroxypropylmethylcellulose, methylcellulose polymer, hydroxyethylcellulose, sodium carboxymethylcellulose, carboxymethylene hydroxyethylcellulose and/or carboxymethyl hydroxyethylcellulose; an acrylic polymer, such as acrylic acid, acrylamide, and maleic anhydride polymers and copolymers; blends of any of the foregoing; or mixtures of any of the foregoing.

In some embodiments, the nanoparticle compositions comprise hydroxypropyl cellulose.

In some embodiments, the nanoparticle compositions comprise hydroxypropylmethyl cellulose (HPMC). HPMC is a semisynthetic, inert, viscoelastic polymer commonly used as an excipient in pharmaceutical formulations.

In certain embodiments, the nanoparticle compositions comprise hydroxypropylmethyl cellulose and the nanoparticles have a weight ratio of dimethyl fumarate to hydroxypropylmethyl cellulose in a range of about 1:1 to about 10:1. In certain embodiments, the nanoparticles have a weight ratio of dimethyl fumarate to hydroxypropylmethyl cellulose in a range of about 1:1 to about 8:1. In certain embodiments, the weight ratio is in a range of about 1:1 to 6:1. In certain embodiments, the weight ratio is in a range of about 1:1 to 4:1. In certain embodiments, the weight ratio is in a range of about 1:1 to 2:1.

In some embodiments, the nanoparticles have a weight ratio of dimethyl fumarate to hydroxypropylmethyl cellulose that is about 5:1.

In certain embodiments, the hydroxypropylmethyl cellulose has a molecular weight in a range of about 2,000 to about 100,000 daltons. In other embodiments, the hydroxypropylmethyl cellulose has a molecular weight in a range of about 10,000 to about 20,000 daltons.

In some embodiments, the hydroxypropylmethyl cellulose is hypromellose 2910. According to the USP, different substitution forms of Hypromellose may be specified by adding a number to the nonproprietary name: e.g., hypromellose 2910, where the first two digits refer to the approximate percent content of the methoxy group (OCH₃) and the second two digits refer to the approximate percent content of the hydroxypropoxy group (OCH₂CH(OH)CH₃), calculated on a dried basis. Therefore, “hypromellose 2910” refers to hydroxypropylmethyl cellulose having a methoxy content of approximately 29% and a hydroxypropoxy content of approximately 10% calculated on a dry basis. In other embodiments, the hydroxypropylmethyl cellulose has a viscosity of about 3 mPas. In other embodiments, the hydroxypropylmethyl cellulose has a viscosity of about 6 mPas.

In some embodiments, the nanoparticle compositions comprise an anionic surfactant. Most anionic surfactants include a hydrocarbon chain, which can be branched, linear, or aromatic, terminating in a highly polar anionic group. The hydrocarbon chain is often comprised of polyether groups. The chains can be ethoxylated (polyethylene oxide-like sequences inserted) to increase the hydrophilic character of a surfactant. Polypropylene oxides may be inserted to increase the lipophilic character of a surfactant. Fluorosurfactants have fluorocarbon chains. Siloxane surfactants have siloxane chains.

The anionic functional group at the head of an anionic surfactant is typically a sulfate, sulfonate, phosphate, or carboxylate moiety. Prominent alkyl sulfate anionic surfactants include ammonium lauryl sulfate, sodium lauryl sulfate (also called sodium dodecyl sulfate or SDS), sodium laureth sulfate (also known as sodium lauryl ether sulfate or SLES), sodium myreth sulfate, docusates, dioctyl sodium sulfosuccinate (DOSS), perfluorooctanesulfonate (PFOS), perfluorobutanesulfonate, and linear alkylbenzene sulfonates (LABs). Anionic surfactants also include alkyl-aryl ether phosphates and alkyl ether phosphatea. Carboxylates are the most common anionic surfactants and comprise the alkyl carboxylates such as sodium stearate. More specialized anionic carboxylate surfactants include sodium lauroyl sarcosinate and carboxylate-based fluorosurfactants such as perfluorononanoate (PFOA), perfluorooctanoate (PFO).

In certain embodiments, the nanoparticles have a weight ratio of dimethyl fumarate to anionic surfactant in a range of about 30:1 to about 300:1. In certain embodiments, the weight ratio is in a range of about 50:1 to about 200:1. In certain embodiments, the weight ratio is in a range of about 50:1 to about 150:1.

In some embodiments, the nanoparticles have a weight ratio of dimethyl fumarate to anionic surfactant that is about 100:1.

In certain embodiments, the anionic surfactant is selected from surfactants having a sulfate, sulfonate, phosphate or carboxylate moiety.

In certain embodiments, the anionic surfactant is docusyl sodium and/or sodium lauryl sulfate.

In some embodiments, the anionic surfactant is dioctyl sodium sulfosuccinate (docusate sodium or DOSS).

In certain embodiments, the nanoparticles have a weight ratio of dimethyl fumarate to DOSS in a range of about 30:1 to about 300:1. In certain embodiments, the weight ratio is in a range of about 50:1 to about 200:1. In certain embodiments, the weight ratio is in a range of about 50:1 to about 150:1. In certain embodiments, the nanoparticles have a weight ratio of dimethyl fumarate to anionic surfactant that is about 100:1.

In some aspects, the present disclosure provides nanoparticle compositions comprising dimethyl fumarate, a stabilizer, and an anionic surfactant.

In other aspects, the present disclosure provides nanoparticle compositions comprising dimethyl fumarate, a non-ionic stabilizer and an ionic stabilizer.

In some embodiments, the non-ionic stabilizer is a cellulose based polymer, such as HPMC. In some embodiments, the ionic stabilizer is an anionic surfactant, such as DOSS.

In some aspects, the present disclosure provides nanoparticle compositions comprising dimethyl fumarate, hydroxypropylmethyl cellulose, and an anionic surfactant.

In some aspects, the present disclosure provides nanoparticle compositions comprising dimethyl fumarate, hydroxypropylmethyl cellulose, and DOSS.

In some embodiments, the hydroxypropylmethyl cellulose is hypromellose 2910. In other embodiments, the hydroxypropylmethyl cellulose has a viscosity of about 3 mPas. In other embodiments, the hydroxypropylmethyl cellulose has a viscosity of about 6 mPas.

In some embodiments, the nanoparticles have a weight ratio of dimethyl fumarate to HPMC to DOSS that is about 10:2:0.1.

In some embodiments, the nanoparticle compositions further comprise a pharmaceutically acceptable carrier or pharmaceutically acceptable vehicle. The carriers or vehicles may comprise one or more diluents or fillers, one or more binders, one or more lubricants, one or more glidants, one or more disintegrants; or a combination thereof.

In certain embodiments, the nanoparticle compositions further comprise a viscosity building agent such as lactose, sucrose, saccharose, a hydrolyzed starch, or a mixture thereof.

In other aspects, the present disclosure provides processes for preparing nanoparticle compositions.

In some embodiments, the nanoparticle compositions are prepared using a roller mill. In other embodiments, the nanoparticle compositions are prepared using a media mill. In other embodiments, the nanoparticle compositions are prepared using a vertical media mill. In further embodiments, the nanoparticle compositions are prepared using a horizontal media mill. In some embodiments, the milling time is about 1 hour (h). In other embodiments, the milling time is about 1.5 h. In other embodiments, the milling time is about 2 h. In other embodiments, the milling time is about 2.5 h. In other embodiments, the milling time is about 3 h.

In some aspects, the present disclosure provides processes for preparing nanoparticle compositions, comprising the steps of milling a mixture comprising micron sized DMF, a polymer and an anionic surfactant to produce a nanoparticle composition. In some embodiments, the nanoparticle compositions are in the form of a suspension.

In some embodiments, the processes further comprise adsorbing the nanoparticles, or a suspension of the nanoparticles, on a carrier to form granules.

In some embodiments, the granules are compressed to form tablets or encapsulated in capsules.

In other embodiments, the processes further comprise spray drying or spray coating or layering the nanoparticles onto a solid support such as cellulose or sugar spheres or onto another pharmaceutically acceptable vehicle.

“Spray Layering” is a procedure where a solution or suspension containing ingredients is sprayed through a nozzle into a fluidized bed containing particles which are coated with a film containing the composition of the solution or suspension as the solvent is removed by the flow of a heated gas. Spray layering typically involves coating an inert core usually comprised of a sugars and starch or cellulosics or combinations thereof. Such cores are typically 20 to 35 mesh in size. Spray Layering is used extensively for applying coatings (finish or enteric) to solid dosage formulations as well as spherical beads containing a drug for use in a capsule or tablet formulation.

In some embodiments, the processes comprise the steps of i) preparing a nanoparticle composition comprising DMF, a surface stabilizer such as HPMC and an anionic surfactant such as DOSS; ii) adding a re-dispersant aid such as sucrose to the composition to obtain a suspension; iii) spray-coating the suspension onto a solid support such as cellulose spheres to form coated spheres; iv) lubricating the coated spheres with a lubricant such as sodium lauryl sulfate; and v) optionally encapsulating the resultant product from the step iv into hard gelatin capsules.

In some embodiments, the solid support comprises cellulose spheres such as microcrystalline cellulose spheres, starch spheres, sugar spheres, sugar-starch spheres, lactose spheres or other pharmaceutically acceptable excipients that are well known in the art.

In some embodiments, with respect to the processes, the polymer may be any polymer or mixtures thereof, as described herein. In some embodiments, the polymer is HPMC.

In some embodiments, with respect to the processes, the anionic surfactant may be any anionic surfactant or mixtures thereof, as described herein. In some embodiments, the anionic surfactant is DOSS.

In certain embodiments, with respect to the processes, the mixture comprising DMF, polymer and the anionic surfactant further comprises one or more viscosity building agents, one or more diluents or fillers, one or more binders, one or more lubricants, one or more glidants, one or more disintegrants, or combinations of any of the foregoing.

In certain embodiments, with respect to the processes, the mixture comprising DMF, polymer and the anionic surfactant further comprises a viscosity building agent such as lactose, sucrose, saccharose, a hydrolyzed starch, or combinations of any of the foregoing.

### Preparation of Nanoparticles

A number of methods are available to produce nanoparticles. For example, spray freezing into liquid (SFL), rapid expansion from a liquefied-gas solution (RESS), and gas antisolvent recrystallization (GAS). RESS and GAS represent two approaches in development based upon supercritical fluid technology (Pathak P, Meziani M J, Sun Y-P., “Supercritical fluid technology for enhanced drug delivery”, Expert Opin. Drug Deliv. 2005(2):747-761). RESS is used for compounds that are soluble in supercritical fluids. The resulting solution is subjected to a rapid reduction in pressure and/or a rapid elevation in temperature, causing the solute to emerge from solution. Under optimal conditions, submicron particles can be generated. The GAS process is used for compounds that are not soluble in supercritical fluids. The compound is first dissolved in an organic solvent and then re-crystallized by admixing with the supercritical fluid. Crystalline nanoparticles can be produced by impinging jet crystallization technology (Panagiotou T, Fisher R J. “Form Nanoparticles via Controlled Crystallization”, Chemical Engineering Progress 2008:33-39).

Other methods include high-pressure homogenization or high-energy wet milling of micron sized drug crystals or particles in a fluid phase consisting essentially of water, yielding drug particles in the nanometer size range (Merisko-Liversidge E, Liversidge G G, Cooper E R. “Nanosizing: a formulation approach for poorly-water-soluble compounds”, Eur. J. Pharm. Sci. 2004, 18: 113-20).

Inclusion of surface modifiers during nanoparticle preparations prevents aggregation and/or Ostwald ripening of the nanoparticles. Surface modifiers are ionic or non-ionic substances capable of wetting the large drug crystals and provide steric and/or ionic stabilization to the resulting nanoparticles.

Exemplary non-ionic stabilizers or surface modifiers include hydroxypropylmethyl cellulose, polyvinylpyrrolidone, Plasdone, polyvinyl alcohol, Pluronics, Tweens and polyethyleneglycols (PEGs).

The ionic surface stabilizers or modifiers are charged organic molecules bearing an ionic bond. The two most described ionic surface stabilizers are the long chain sulfonic acid salts sodium lauryl sulfate and dioctyl sodium sulfosuccinate (DOSS).

Examples of surface stabilizers are disclosed in U.S. Pat. No. 5,145,684. Typically, 0%450% (wt % of drug) of a nonionic surface stabilizer and 0.1%-5% of an ionic surface stabilizer (wt % of drug) achieve maximal particle size stabilization.

### Pharmaceutical Compositions

The present disclosure relates to pharmaceutical compositions comprising a therapeutically effective amount of dimethyl fumarate-containing nanoparticles or nanoparticle compositions as disclosed herein and a pharmaceutically acceptable vehicle or carrier (also known as a pharmaceutically acceptable excipient). Depending on the type of pharmaceutical composition, the pharmaceutically acceptable vehicle or carrier may be chosen from any one of a combination of carriers known in the art. The choice of the pharmaceutically acceptable vehicle or carrier depends upon the pharmaceutical form and the desired method of administration to be used. In some embodiments, the pharmaceutical compositions are formulated in unit dosage forms for ease of administration and uniformity of dosage. A “unit dosage form” refers to a physically discrete unit of therapeutic agent appropriate for the patient to be treated. It will be understood, however, that the total daily dose of the therapeutic agent will typically be decided by the attending physician within the scope of sound medical judgment.

In certain embodiments, the pharmaceutical composition may be selected from any one or more known solid form, such as a solid oral dosage form. Solid dosage forms may be employed in numerous embodiments for the pharmaceutical compositions. In some embodiments, solid dosage forms for oral administration include capsules, tablets, pills, powders, and granules. Solid oral dosage forms, including capsules, tablets, pills, and granules, may be of any shape suitable for oral administration of a drug such as spheroidal, cube-shaped, oval, or ellipsoidal. In such solid dosage forms, the active compound is mixed with at least one pharmaceutically acceptable vehicle or carrier, such as for example sodium citrate or dicalcium phosphate. The solid dosage forms may also include one or more of: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) dissolution retarding agents such as paraffin; f) absorption accelerators such as quaternary ammonium compounds; g) wetting agents such as, for example, cetyl alcohol and glycerol monostearate; h) absorbents such as kaolin and bentonite clay; and/or i) lubricants such as talc, calcium stearate, magnesium stearate, solid polyethylene glycols and sodium lauryl sulfate. The solid dosage forms may also comprise buffering agents. They may also optionally contain opacifying agents and can also be of a composition such that they release the active ingredient(s) only in a certain part of the intestinal tract, optionally, in a delayed manner. Remington's Pharmaceutical Sciences, Sixteenth Edition, E. W. Martin (Mack Publishing Co., Easton, Pa., 1980) discloses various vehicles or carriers used in formulating pharmaceutical compositions and known techniques for the preparation thereof. Solid dosage forms of pharmaceutical compositions can also be prepared with coatings and shells such as enteric coatings and other coatings known in the art.

In certain aspects, the pharmaceutical composition (e.g., solid oral dosage forms) may be formed by compressing the nanoparticles, optionally combined with one or more pharmaceutically acceptable carriers, as disclosed herein. In certain embodiments, the pharmaceutical compositions (e.g., solid oral dosage forms) may comprise compressed nanoparticles, optionally combined with one or more pharmaceutically acceptable carriers, as described herein. In other embodiments, the pharmaceutical composition may comprise a matrix system. In various embodiments, the matrix system may comprise compressed nanoparticles, optionally combined with one or more pharmaceutically acceptable carriers, as disclosed herein. Matrix systems are well-known in the art as described, for example, in “Handbook of Pharmaceutical Controlled Release Technology,” ed. Wise, Marcel Dekker, Inc. (2000) and “Treatise on Controlled Drug Delivery, Fundamentals, Optimization, and Applications,” ed. Kydonieus, Marcel Dekker, Inc. (1992). In various embodiments, the pharmaceutical compositions formed from compressed nanoparticles (or comprising compressed nanoparticles) can have dissolution rates faster than pharmaceutical compositions formed from particles larger than the nanoparticles.

Also disclosed herein are methods for the treatment of the disorders disclosed herein. The dimethyl fumarate nanoparticle compositions, and pharmaceutical compositions comprising them, may be administered using any amount, any form of pharmaceutical composition and any route of administration effective for the treatment. After formulation with an appropriate pharmaceutically acceptable vehicle or carrier in a desired dosage, as known by those of skill in the art, the pharmaceutical compositions can be administered to humans and other animals orally, rectally, parenterally, intravenously, intracisternally, intravaginally, intraperitoneally, topically (as by powders, ointments, or drops), bucally, as an oral or nasal spray, or the like, depending on the location and severity of the condition being treated. In certain embodiments, the dimethyl fumarate may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject.

As described above, the present disclosure provides pharmaceutical compositions comprising nanoparticles of DMF and/or nanoparticle compositions described herein.

In some embodiments, the pharmaceutical compositions are in a parenteral dosage form containing a therapeutically effective amount of dimethyl fumarate.

In some embodiments, the pharmaceutical compositions are in an oral dosage form containing a therapeutically effective amount of dimethyl fumarate.

In some embodiments, the pharmaceutical compositions are in a form of a tablet dosage form, a pill dosage form, a capsule dosage form, a sustained release formulation dosage form, a liquid dosage form or a powder dosage form.

In some embodiments, the pharmaceutical compositions further comprise a pharmaceutically acceptable vehicle or carrier. The vehicles or carriers may be as defined herein and/or may comprise one or more diluents or fillers, one or more binders, one or more lubricants, one or more glidants, one or more disintegrants; or a mixture thereof.

Pharmaceutically acceptable vehicles or carriers that may be a part of the compositions disclosed herein include, but are not limited to, at least one of: ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor, phosphate buffered saline (PBS), and combinations of any of the foregoing.

In some embodiments, the pharmaceutical compositions comprise a pharmaceutically acceptable vehicle or carrier and nanoparticles are adsorbed onto the surface of the vehicle or carrier.

In certain embodiments, the vehicle or carrier comprises lactose monohydrate, microcrystalline cellulose, crospovidone, or mixtures thereof.

### Therapeutic Uses

The dimethyl fumarate nanoparticle compositions disclosed herein may be used to treat diseases, disorders, conditions, and/or symptoms of any disease or disorder for which DMF and/or MMF is known to provide, or is later found to provide, therapeutic benefit. DMF and MMF are known to be effective in treating psoriasis, multiple sclerosis, an inflammatory bowel disease, asthma, chronic obstructive pulmonary disease, and arthritis. Hence, the dimethyl fumarate nanoparticle compositions disclosed herein may be used to treat any one or more of the foregoing diseases and disorders. The underlying etiology of any of the foregoing diseases may have a multiplicity of origins. Further, in certain embodiments, a therapeutically effective amount of one or more of the dimethyl fumarate nanoparticle compositions may be administered to a patient, such as a human, as a preventative measure against various diseases or disorders. Thus, a therapeutically effective amount of one or more of the dimethyl fumarate nanoparticle compositions may be administered as a preventative measure to a patient having a predisposition for and/or history of immunological, autoimmune, and/or inflammatory diseases including psoriasis, asthma, chronic obstructive pulmonary disease, cardiac insufficiency including left ventricular insufficiency, myocardial infarction and angina pectoris, mitochondrial and neurodegenerative diseases (such as Parkinson's disease, Alzheimer's disease, Huntington's disease, retinopathia pigmentosa and mitochondrial encephalomyopathy), transplantation rejection, autoimmune diseases including multiple sclerosis, ischemia and reperfusion injury, AGE-induced genome damage, inflammatory bowel diseases such as Crohn's disease and ulcerative colitis, and NF-κB mediated diseases.

### Psoriasis

Psoriasis is characterized by hyperkeratosis and thickening of the epidermis as well as by increased vascularity and infiltration of inflammatory cells in the dermis. Psoriasis vulgaris manifests as silvery, scaly, erythematous plaques on typically the scalp, elbows, knees, and buttocks. Guttate psoriasis occurs as tear-drop size lesions.

Fumaric acid esters are recognized for the treatment of psoriasis and dimethyl fumarate is approved for the systemic treatment of psoriasis in Germany (Mrowietz and Asadullah, Trends Mol Med (2005), 11(1): 43-48; and Mrowietz et al., Br J Dermatology (1999), 141: 424-429).

Efficacy of the dimethyl fumarate nanoparticle compositions for treating psoriasis can be determined using animal models and in clinical trials.

### Inflammatory Arthritis

Inflammatory arthritis includes diseases such as rheumatoid arthritis, juvenile rheumatoid arthritis (juvenile idiopathic arthritis), psoriatic arthritis, and ankylosing spondylitis, among others. The pathogenesis of immune-mediated inflammatory diseases including inflammatory arthritis is believed to involve TNF and NK-κB signaling pathways (Tracey et al., Pharmacology & Therapeutics (2008), 117: 244-279). Dimethyl fumarate has been shown to inhibit TNF and inflammatory diseases, including inflammatory arthritis, are believed to involve TNF and NK-κB signaling. Therefore, dimethyl fumarate may be useful in treating inflammatory arthritis (Lowewe et al., J Immunology (2002), 168: 4781-4787).

The efficacy of the dimethyl fumarate nanoparticle compositions for treating inflammatory arthritis can be determined using animal models and in clinical trials.

### Multiple Sclerosis

Multiple sclerosis (MS) is an inflammatory autoimmune disease of the central nervous system caused by an autoimmune attack against the insulating axonal myelin sheaths of the central nervous system. Demyelination leads to the breakdown of conduction and to severe disease with destruction of local axons and irreversible neuronal cell death. The symptoms of MS are highly varied, with each individual patient exhibiting a particular pattern of motor, sensible, and sensory disturbances. MS is typified pathologically by multiple inflammatory foci, plaques of demyelination, gliosis, and axonal pathology within the brain and spinal cord, all of which contribute to the clinical manifestations of neurological disability (see e.g., Wingerchuk, Lab Invest (2001), 81: 263-281; and Virley, NeuroRx (2005), 2(4): 638-649). Although the causal events that precipitate MS are not fully understood, evidence implicates an autoimmune etiology together with environmental factors, as well as specific genetic predispositions. Functional impairment, disability, and handicap are expressed as paralysis, sensory and octintive disturbances, spasticity, tremor, a lack of coordination, and visual impairment, any one of which negatively impacts the quality of life of the individual. The clinical course of MS can vary from individual to individual, but invariably the disease can be categorized in three forms: relapsing-remitting, secondary progressive, and primary progressive.

Studies support the efficacy of fumaric acid esters for treating MS and dimethyl fumarate has been approved in the US for such treatment (Schimrigk et al., Eur J Neurology (2006), 13: 604-610; and Wakkee and Thio, Current Opinion Investigational Drugs (2007), 8(11): 955-962).

Assessment of MS treatment efficacy in clinical trials can be accomplished using tools such as the Expanded Disability Status Scale and the MS Functional, as well as magnetic resonance imaging, lesion load, biomarkers, and self-reported quality of life. Animal models of MS shown to be useful to identify and validate potential therapeutics include experimental autoimmune/allergic encephalomyelitis (EAE) rodent models that simulate the clinical and pathological manifestations of MS and nonhuman primate EAE models.

The efficacy of the dimethyl fumarate nanoparticle compositions for treating MS can be determined using animal models and in clinical trials.

### Inflammatory Bowel Disease (Crohn's Disease, Ulcerative Colitis)

Inflammatory bowel disease (IBD) is a group of inflammatory conditions of the large intestine, and in some cases the small intestine, that includes Crohn's disease and ulcerative colitis. Crohn's disease, which is characterized by areas of inflammation with areas of normal lining in between, can affect any part of the gastrointestinal tract from the mouth to the anus. The main gastrointestinal symptoms are abdominal pain, diarrhea, constipation, vomiting, weight loss, and/or weight gain. Crohn's disease can also cause skin rashes, arthritis, and inflammation of the eye. Ulcerative colitis is characterized by ulcers or open sores in the large intestine or colon. The main symptom of ulcerative colitis is typically constant diarrhea with mixed blood of gradual onset. Other types of intestinal bowel disease include collagenous colitis, lymphocytic colitis, ischaemic colitis, diversion colitis, Behcet's colitis, and indeterminate colitis.

Fumaric acid esters are inhibitors of NF-κB activation and therefore may be useful in treating inflammatory diseases such as Crohn's disease and ulcerative colitis (Atreya et al., J Intern Med (2008), 263(6): 591-596).

The efficacy of the dimethyl fumarate nanoparticle compositions for treating inflammatory bowel disease can be evaluated using animal models and in clinical trials. Useful animal models of inflammatory bowel disease are known.

### Asthma

Asthma is reversible airway obstruction in which the airway occasionally constricts, becomes inflamed, and is lined with an excessive amount of mucus. Symptoms of asthma include dyspnea, wheezing, chest tightness, and cough. Asthma episodes may be induced by airborne allergens, food allergies, medications, inhaled irritants, physical exercise, respiratory infection, psychological stress, hormonal changes, cold weather, or other factors.

As an inhibitor of NF-κB activation and as shown in animal studies (Joshi et al., U.S. Patent Application Publication No. 2007/0027076) fumaric acid esters may be useful in treating pulmonary diseases such as asthma and chronic obstructive pulmonary disorder.

The efficacy of the dimethyl fumarate nanoparticle compositions for treating asthma can be assessed using animal models and in clinical trials.

### Chronic Obstructive Pulmonary Disease

Chronic obstructive pulmonary disease (COPD), also known as chronic obstructive airway disease, is a group of diseases characterized by the pathological limitation of airflow in the airway that is not fully reversible, and includes conditions such as chronic bronchitis, emphysema, as well as other lung disorders such as asbestosis, pneumoconiosis, and pulmonary neoplasms (see, e.g., Barnes, Pharmacological Reviews (2004), 56(4): 515-548). The airflow limitation is usually progressive and associated with an abnormal inflammatory response of the lungs to noxious particles and gases. COPD is characterized by a shortness of breath that can last for months or years, possibly accompanied by wheezing, and a persistent cough with sputum production. COPD is most often caused by tobacco smoking, although it can also be caused by other airborne irritants such as coal dust, asbestos, urban pollution, or solvents. COPD encompasses chronic obstructive bronchiolitis with fibrosis and obstruction of small airways, and emphysema with enlargement of airspaces and destruction of lung parenchyma, loss of lung elasticity, and closure of small airways.

The efficacy of the dimethyl fumarate nanoparticle compositions for treating chronic obstructive pulmonary disease may be assessed using animal models of chronic obstructive pulmonary disease and in clinical studies. For example, murine models of chronic obstructive pulmonary disease are known.

### Neurodegenerative Disorders

Neurodegenerative diseases such as Parkinson's disease, Alzheimer's disease, Huntington's disease and amyoptrophic lateral sclerosis are characterized by progressive dysfunction and neuronal death. NF-κB inhibition has been proposed as a therapeutic target for neurodegenerative diseases (Camandola and Mattson, Expert Opin Ther Targets (2007), 11(2): 123-32).

The efficacy of the dimethyl fumarate nanoparticle compositions for treating neurodegenerative disorders may be assessed using animal and human models of neurodegenerative disorders and in clinical studies.

### Parkinson's Disease

Parkinson's disease is a slowly progressive degenerative disorder of the nervous system characterized by tremor when muscles are at rest (resting tremor), slowness of voluntary movements, and increased muscle tone (rigidity). In Parkinson's disease, nerve cells in the basal ganglia (e.g., the substantia nigra) degenerate, and thereby reduce the production of dopamine and the number of connections between nerve cells in the basal ganglia. As a result, the basal ganglia are unable to control smooth muscle movements and coordinate changes in posture as normal, leading to tremor, incoordination, and slowed, reduced movement (bradykinesia) (Blandini, et al., Mol. Neurobiol. (1996), 12: 73-94).

The efficacy of the dimethyl fumarate nanoparticle compositions for treating Parkinson's disease may be assessed using animal and human models of Parkinson's disease and in clinical studies.

### Alzheimer's Disease

Alzheimer's disease is a progressive loss of mental function characterized by degeneration of brain tissue, including loss of nerve cells and the development of senile plaques and neurofibrillary tangles. In Alzheimer's disease, parts of the brain degenerate, destroying nerve cells and reducing the responsiveness of the maintaining neurons to neurotransmitters. Abnormalities in brain tissue consist of senile or neuritic plaques (e.g., clumps of dead nerve cells containing an abnormal, insoluble protein called amyloid) and neurofibrillary tangles, twisted strands of insoluble proteins in the nerve cell.

The efficacy of the dimethyl fumarate nanoparticle compositions for treating Alzheimer's disease may be assessed using animal and human models of Alzheimer's disease and in clinical studies.

### Huntington's Disease

Huntington's disease is an autosomal dominant neurodegenerative disorder in which specific cell death occurs in the neostriatum and cortex (Martin, N Engl J Med (1999), 340: 1970-80). Onset usually occurs during the fourth or fifth decade of life, with a mean survival at age of onset of 14 to 20 years. Huntington's disease is universally fatal, and there is no effective treatment. Symptoms include a characteristic movement disorder (Huntington's chorea), cognitive dysfunction, and psychiatric symptoms. The disease is caused by a mutation encoding an abnormal expansion of CAG-encoded polyglutamine repeats in the protein, huntingtin.

The efficacy of the dimethyl fumarate nanoparticle compositions for treating Huntington's disease may be assessed using animal and human models of Huntington's disease and in clinical studies.

### Amyotrophic Lateral Sclerosis

Amyotrophic lateral sclerosis (ALS) is a progressive neurodegenerative disorder characterized by the progressive and specific loss of motor neurons in the brain, brain stem, and spinal cord (Rowland and Schneider, N Engl J Med (2001), 344: 1688-1700). ALS begins with weakness, often in the hands and less frequently in the feet that generally progresses up an arm or leg. Over time, weakness increases and spasticity develops characterized by muscle twitching and tightening, followed by muscle spasms and possibly tremors. The average age of onset is 55 years, and the average life expectancy after the clinical onset is 4 years. The only recognized treatment for ALS is riluzole, which can extend survival by only about three months.

The efficacy the dimethyl fumarate nanoparticle compositions for treating ALS may be assessed using animal and human models of ALS and in clinical studies.

### Other Diseases

Other diseases and conditions for which the dimethyl fumarate nanoparticle compositions can be useful in treating include: rheumatica, granuloma annulare, lupus, autoimmune carditis, eczema, sarcoidosis, autoimmune diseases including acute disseminated encephalomyelitis, Addison's disease, alopecia greata, ankylosing spondylitis, antiphospholipid antibody syndrome, autoimmune hemolytic anemia, autoimmune hepatitis, autoimmune inner ear disease, bullous pemphigoid, Behcet's disease, celiac disease, Chagas disease, chronic obstructive pulmonary disease, Crohn's disease, dermatomyositis, diabetes mellitus type I, endometriosis, Goodpasture's syndrome, Graves' disease, Guillain-Barre syndrome, Hashimoto's disease, hidradenitis suppurativea, Kawasaki disease, IgA neuropathy, idiopathic thrombocytopenic purpura, interstitial cystitis, lupus erythematosus, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, rheumatoid arthritis, schizophrena, scleroderma, Sjogren's syndrome, stiff person syndrome, temporal arteritis, ulcerative colitis, vasculitis, vitiligo, Wegener's granulomatosis, optic neuritis, neuromyelitis optica, subacute necrotizing myelopathy, balo concentric sclerosis, transverse myelitis, susac syndrome, central nervous system vasculitis, neurosarcoidosis, Charcott-Marie-Tooth Disease, progressive supranuclear palsy, neurodegeneration with brain iron accumulation, pareneoplastic syndromes, primary lateral sclerosis, Alper's Disease, monomelic myotrophy, adrenal leukodystrophy, Alexander's Disease, Canavan disease, childhood ataxia with central nervous system hypomyelination, Krabbe Disease, Pelizaeus-Merzbacher disease, Schilders Disease, Zellweger's syndrome, Sjorgren's Syndrome, human immunodeficiency viral infection, hepatitis C viral infection, herpes simplex viral infection and tumors.

### EXAMPLES

The following examples are included to demonstrate certain embodiments of the present disclosure. It should be appreciated by those of skill in the art that the techniques disclosed in the examples which follow represent techniques discovered by the inventors to function well in the practice of the subject matter of the present disclosure, and thus can be considered to constitute modes for its practice. However, those of skill in the art should, in light of the present disclosure, appreciate that many changes can be made to the specific embodiments disclosed herein and still obtain a like or similar result without departing from the spirit and scope of the subject matter of the present disclosure.

### Preparation of Aqueous Suspension of DMF Nanoparticles

### Example 1

### Preparation of DMF Nanoparticles Using Media Mill Process

### Media Milling of 20% Dimethyl Fumarate Suspension with 4% Hypromellose 2910, 3 mPas and 0.2% Docusate Sodium in a Vertical Mill with Polystyrene-Divinylbenzene Milling Media

A vertical, high speed media batch mill was assembled by attaching an agitator and chamber lid to the motor shaft. An aqueous suspension containing 9.56 g of dimethyl fumarate having a median particle size of about 400 μm, 1.912 g of hydroxypropylmethyl cellulose (Hypromellose 2910, viscosity of 3 mPas), 0.096 g of the anionic surfactant docusate sodium, 36.232 g of de-ionized water and 51.850 g of polystyrene-divinylbenzene milling media was added to a 100 mL stainless steel milling chamber which was then clamped to the lid, such that the agitator shaft protruded into the chamber through the lid. The jacketed chamber was cooled to 5° C. with attached chiller lines. The motor controller was set for the specified agitator speed of 3,500 rpm and time of 1 hour. After milling was completed, the chamber was opened and the contents transferred to a centrifuge tube insert, fitted with a screen mesh. The tube was then centrifuged to collect the dispersion below the screen (which retained the media). The nanoparticulate suspension was then characterized using an Olympus B51 microscope and a Horiba LA-950V2 particle size analyzer.

A representative batch record from the media mill process is shown in Table 1.

TABLE 1
Representative Batch Record for Media Mill Composition

| | |
|---|---|
| Composition | 20% DMF, 4% HPMC, viscosity of 3 mPas, 0.2% DOSS |
| Batch Size | 47.8 g |

| | % (w/w) | Quantity (g) | Component |
|---|---|---|---|
| API | 20.0 | 9.56 | DMF |
| Stabilizer #1 | 4.0 | 1.912 | HPMC |
| | | | (viscosity 3 mPas) |
| Stabilizer #2 | 0.2 | 0.096 | DOSS |
| Water | 75.8 | 36.232 | DI |
| Media Load % (v/v) | 85 | 51.850 | Polystyrene- |
| | | | divinylbenzene |

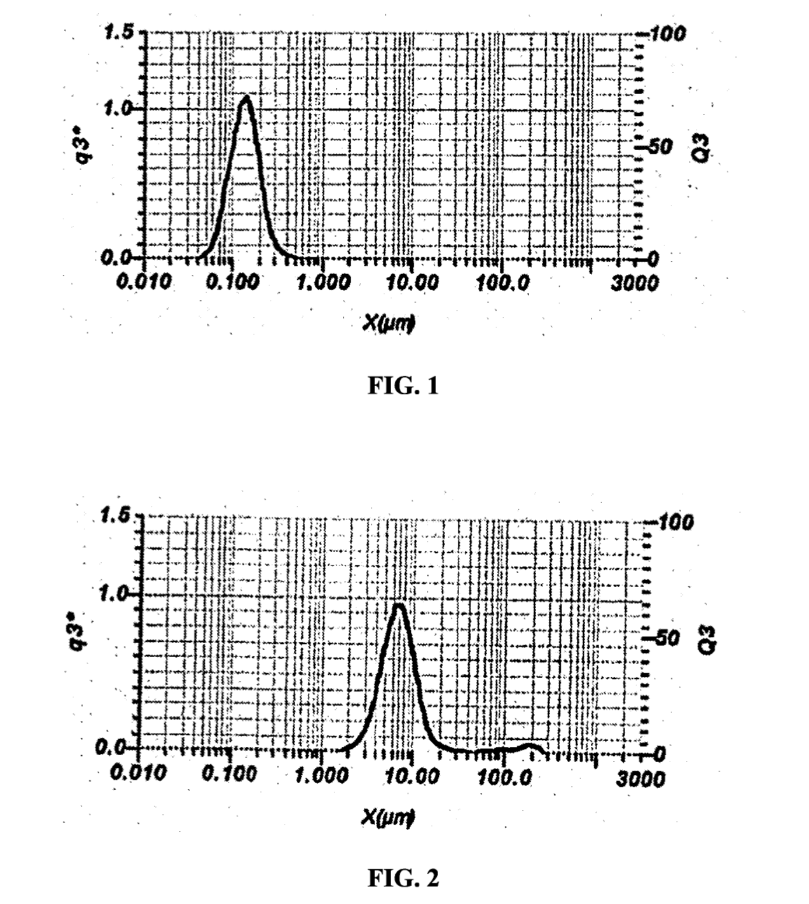

The high energy milling with polystyrene media resulted in a homogeneous nanodispersion. FIG. 1 shows the particle size distribution of the nanoparticulate suspension. The median particle size (volume basis) was measured to be 134 nm and d₉₀ value was measured to be 216 nm.

### Example 2

### Media Milling of 20% Dimethyl Fumarate Suspension with 4% Plasdone S-630 and 0.2% Docusate Sodium in a Vertical Mill with Polystyrene-Divinylbenzene Milling Media

A vertical, high speed media batch mill was assembled by attaching an agitator and chamber lid to the motor shaft. An aqueous suspension containing 21.3 g of dimethyl fumarate having a median particle size of about 400 μm, 4.3 g of S-630 copovidone which is 60:40 copolymer of N-Vinyl-2-Pyrrolidone and vinyl acetate, 0.21 g of the anionic surfactant docusate sodium, 80.7 g of de-ionized water and 129.6 g of polystyrene-divinylbenzene milling media was added to a 250 mL stainless steel milling chamber which was then clamped to the lid, such that the agitator shaft protruded into the chamber through the lid. The jacketed chamber was cooled to 5° C. with attached chiller lines. The motor controller was set for the specified agitator speed of 3,500 rpm. The milling was conducted over a period of 5 hours, during which time samples were periodically removed by withdrawing a 0.2 ml sample using a 26 ga needle. The suspension was then characterized using an Olympus B51 microscope and a Horiba LA-950V2 particle size analyzer. However, the above method failed to produce the desired nanoparticle composition after the 5 hours of milling, as the mean particle size (volume basis) was measured to be 2.03 μm and d₉₀ value was measured to be 3.35 μm.

### Example 3

### Media Milling of 20% Dimethyl Fumarate Suspension with 4% Poloxamer 407 and 0.2% Docusate Sodium in a Vertical Mill with Polystyrene-Divinylbenzene Milling Media

A vertical, high speed media batch mill was assembled by attaching an agitator and chamber lid to the motor shaft. An aqueous suspension containing 1.7 g of dimethyl fumarate having a median particle size of about 400 μm, 0.35 g of Poloxamer 407 which is a triblock copolymer of polyethylene glycol and polypropylene glycol, 0.017 g of the anionic surfactant docusate sodium, 6.5 g of de-ionized water and 12.1 g of polystyrene-divinylbenzene milling media was added to a 22 mL stainless steel milling chamber which was then clamped to the lid, such that the agitator shaft protruded into the chamber through the lid. The jacketed chamber was cooled to 5° C. with attached chiller lines. The motor controller was set for the specified agitator speed of 5,000 rpm and time of 2 hours. After milling was completed, a 0.2 ml sample was withdrawn using a 26 ga needle. The suspension was then characterized using an Olympus B51 microscope. However, the above method failed to produce the desired nanoparticle composition as the microscope showed aggregated particles of approximately 1-5 μm diameter.

### Example 4

### Media Milling of 20% Dimethyl Fumarate Suspension with 4% Hypromellose 2910, 3 mPas and 0.2% Docusate Sodium in a Netzsch DeltaVita 15-300 Mill with YTZ (Yttria Stabilized Tetragonal Zirconia) Media

An aqueous suspension containing 60 g of dimethyl fumarate of having median particle size of about 400 μm, 12 g of hydroxypropylmethyl cellulose (Hypromellose 2910, viscosity of 3 mPas), 0.6 g of docusate sodium, 227.4 g of de-ionized water and 438 g of YTZ-500 media (Yttria stabilized tetragonal zirconia, Tosoh Corporation, Tokyo, Japan) was loaded into a 150 mL chamber of a Netzsch DeltaVita model 15-300, a high speed horizontal stainless steel recirculation mill that utilizes a scalable GMP-capable platform. This mill incorporated an internal screen and the product was recirculated through a collection vessel until milling was completed. The jacketed chamber was cooled to 5° C. with attached chiller lines. The motor controller was set for the specified agitator speed of 3,000 rpm (10.766 m/sec). The composition was recirculated from a holding vessel, into the mill and returned to the vessel until there was significant particle size reduction. The milled suspension was then recovered by pumping from the milling chamber. The suspension was evaluated at successive time points using optical microscopy (OM) and particle size distribution (PSD).

FIG. 2 shows the particle size distribution of the nanoparticulate suspension after 5 minutes of milling. The median particle size (volume basis) was measured to be 6.9 μm and d₉₀ value was measured to be 12.9 μm.

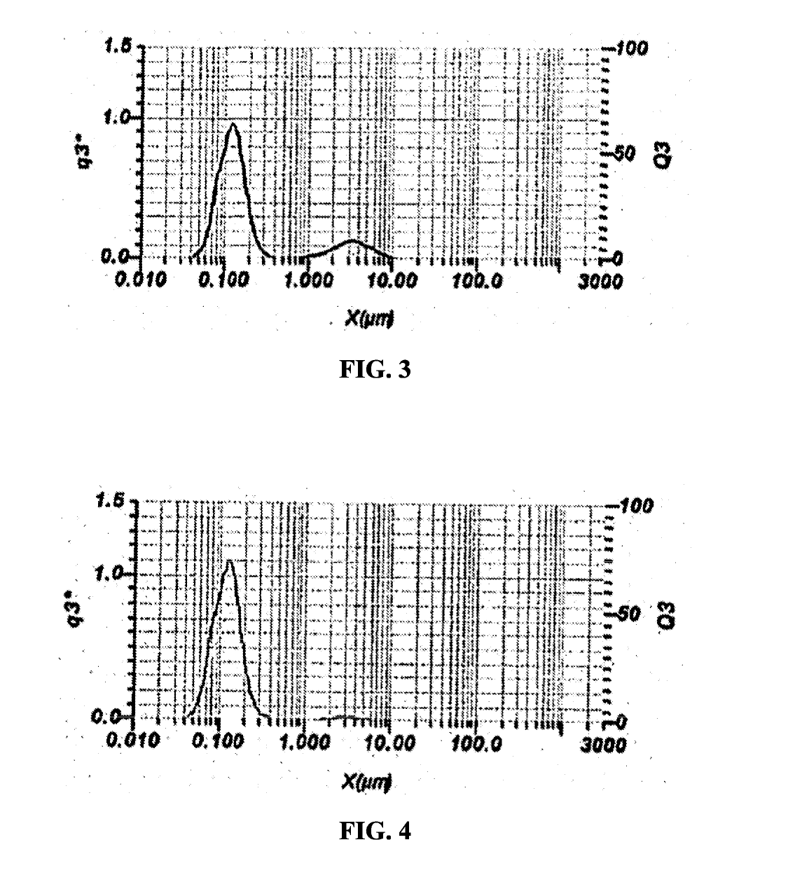

FIG. 3 shows the particle size distribution of the nanoparticulate suspension after 30 minutes of milling. The median particle size (volume basis) was measured to be 130 nm and d₉₀ value was measured to be 2.52 μm.

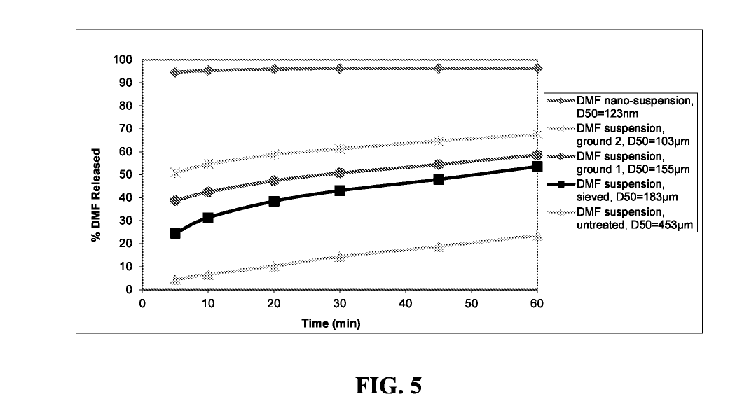

FIG. 4 shows the particle size distribution of the nanoparticulate suspension after 2 hours and 30 minutes of milling. The median particle size (volume basis) was measured to be 123 nm and d₉₀ value was measured to be 197 nm.

A representative batch record from the recirculation mill process is shown in Table 2.

TABLE 2
Batch Record for Example 4 Composition

| | |
|---|---|
| Composition | 20% DMF, 4% HPMC, viscosity of 3 mPas, 0.2% DOSS |
| Batch Size | 300.0 g |

| | % (w/w) | Quantity (g) | Component |
|---|---|---|---|
| API | 20.0 | 60.0 | DMF |
| Stabilizer #1 | 4.0 | 12.0 | HPMC |
| | | | (viscosity 3 mPas) |
| Stabilizer #2 | 0.2 | 0.6 | DOSS |
| Water | 75.8 | 227.4 | DI |
| Media Load % (v/v) | 80 | 438.0 | YTZ-500 |

### Example 5

### Evaluation of DMF Nanoparticulate Suspension Using Optical Microscopy (OM)

The optical microscopy photomicrographs of the suspensions provided by the present disclosure were taken using an Olympus BX51 system equipped with an oil immersion 100× objective (1,000× magnification). A calibration bar (from 1 μm to 100 μm) was set as a comparator on each photomicrograph.

Dispersions were initially evaluated by microscopy after one day of milling at RT. Particle distributions were considered satisfactory if they appeared relatively homogeneous, discrete and consisted of predominately sub-micron particles.

### Example 6

### Dimethyl Fumarate Nanoparticulate Suspension Stability Testing

The stability of the nanoparticulate suspension described in Example 1 was tested as follows. Samples of the suspension were diluted with water to form 1% and 20% DMF concentrations, and both were placed on stability at 5° C. and room temperature. After one month, the suspensions had maintained physical stability. The nanoparticulate suspension was then characterized using an Olympus B51 microscope and a Horiba LA-950V2 particle size analyzer. Little change occurred in both 1 and 20% concentrations at 5° C. and room temperature.

The stability data of the Example 1 Composition is shown in Table 3.

TABLE 3
Stability Data of Example 1 Composition

| | | | Stability Particle Size | | | | | |
|---|---|---|---|---|---|---|---|---|
| | | | 5° C. | | | RT | | |
| | | | Me- | | | Me- | | |
| | Time | | dian | Mean | D 90 | dian | Mean | D 90 |
| Composition | Point | | (nm) | (nm) | (nm) | (nm) | (nm) | (nm) |
| Example 1 | Initial 20% | | — | — | — | 134 | 143 | 216 |
| Composition: | 7 d | 20% | — | photo | stable | — | — | — |
| 20% DMF, | | 1% | — | photo | stable | — | — | — |
| 4% HPMC | 16 d | 20% | 143 | 152 | 227 | 142 | 151 | 226 |
| 0.2% DOSS | | 1% | 150 | 162 | 249 | 138 | 148 | 223 |
| | 28 d | 20% | 145 | 157 | 240 | 141 | 151 | 227 |
| | | 1% | 146 | 158 | 245 | 142 | 153 | 236 |

### Example 7

The stability of the nanoparticulate suspension described in Example 4 was tested at 13 and 32 days. Samples of the suspension were diluted with water to form 1% and 20% Dimethyl fumarate concentrations, and both were placed on stability at 5° C. and room temperature. All compositions were found to be stable.

The stability data of the Example 4 Composition is shown in Table 4.

TABLE 4
Stability Data for Example 4 Composition

| | | | Particle Size @ 5° C. | | | | Particle Size @ RT | | | |
|---|---|---|---|---|---|---|---|---|---|---|
| | Time | | D 10 | Median | Mean | D 90 | D 10 | Median | Mean | D 90 |
| | Point | | (nm) | (nm) | (nm) | (nm) | (nm) | (nm) | (nm) | (nm) |
| Example 4: | 5 | min. | — | — | — | — | 4000 | 6913 | 13472 | 12856 |
| 20% DMF | 30 | min. | — | — | — | — | 79 | 130 | 625 | 2516 |
| 4% HPMC- | 1 | hr. | — | — | — | — | 81 | 133 | 307 | 251 |
| (viscosity | 2 | hr. | — | — | — | — | 79 | 127 | 794 | 213 |
| 3 mPas) | 2:30 | hr. | — | — | — | — | 77 | 123 | 175 | 197 |
| 0.2% DOSS | | | | | | | | | | |

Short-Term Stability

| | | | | | | | | | | |
|---|---|---|---|---|---|---|---|---|---|---|
| 20% | 13 | d | 86 | 144 | 296 | 248 | 83 | 138 | 333 | 234 |
| 1% | 13 | d | 90 | 154 | 506 | 289 | 92 | 158 | 915 | 367 |

### Example 8

The chemical stability of the nanoparticulate suspension described in Example 4 was tested at different time points. Samples of the suspension were diluted with water to form 1% and 20% dimethyl fumarate concentrations, and both concentrations were tested for their degradation to MMF.

The stability data of Example 4 Composition is shown in Table 5.

TABLE 5
Stability Data for Example 4 Composition

| | | Time Point | MMF Composition |
|---|---|---|---|
| Concentration | Conditions | (weeks) | (% wt/wt) |
| 1% | 2-8° C. | 0 | 0.10 |
| | | 1 | 0.10 |
| | | 2 | 0.12 |
| | | 4 | 0.13 |
| 20% | 2-8° C. | 0 | 0.08 |
| | | 1 | 0.07 |
| | | 2 | 0.07 |
| | | 4 | 0.08 |
| 20% | 25° C./ | 0 | 0.08 |
| | 60% RH | 1 | 0.08 |
| | | 2 | 0.08 |
| | | 4 | 0.10 |

No significant degradation was observed for the 1% suspension at 2-8° C.; or for the 20% suspension at 2-8° C. and at 25° C. with 60% relative humidity (RH).

### Example 9

### Representative Method for Capsule Preparation

The nanoparticle suspension from Example 1 or 4 is sprayed onto microcrystalline cellulose (celphere) substrate spheres to a target weight gain of about 450 wt. % using a 24″ Glatt Wurster coating column. The drug-coated beads are screened through a nominal 864 μm and 1,532 μm sieve screen using a 30″ Sweco sieve to remove any fines (<864 μm) and aggregates (>1,532 μm) generated, respectively, during the coating process. The drug-coated beads between approximately 864 μm and approximately 1,532 μm are blended with jet-milled sodium lauryl sulfate using a 300-L Bohle blender. The blended, coated beads from the blender are discharged into batches or subbatches as desired. A subbatch of the blended beads is filled into hard gelatin capsules to the target fill weight to provide 240 mg dose capsules.

### Example 10

### Dissolution Testing

Dimethyl fumarate (DMF) in solid powder form was obtained from Tokyo Chemical Industry Co., Ltd., Tokyo, Japan. A portion of the DMF was sieved through an 80 mesh screen to reduce particle size. Two separate portions of the sieved material were then ground using a mortar and pestle to further reduce particle size.

DMF suspensions were prepared at 200 mg/ml by mixing the untreated, sieved and both ground DMF materials in an aqueous vehicle containing 0.5% methyl cellulose (1500 cps) and 0.1% Tween 80 by stirring.

Particle size distribution of dry DMF powder was measured using an image-based SympaTec QicPic particle size analyzer equipped with the RODOS dispersion module and VIBRI OASIS/L feeder. Particle size distribution of DMF nanoparticle suspensions were measured using a Horiba Laser Scattering Particle Size Distribution Analyzer LA-950. Measured Dv50 values (median particle size based on a volumetric particle size distribution) for samples are provided in Table 6 and in FIG. 5.

Dissolution testing was performed using a USP apparatus II (paddle) at 50 rpm. The dissolution medium contained 500 mL of 50 mM sodium phosphate buffer, pH 6.8, equilibrated to 37° C. A 1 mL volume of 200 mg/mL DMF suspension was added to each dissolution vessel. Samples were collected at 5, 10, 20, 30, 45 and 60 minutes, and assayed for DMF content by reverse-phase HPLC with UV detection at 210 nm.

The dissolution results are provided in Table 6 and in FIG. 5. The results show that dissolution of the nano-particle suspension is much faster compared with the suspensions of ground, sieved or untreated material at concentrations of 200 mg/ml. Over 95% the DMF nano-particle suspension dissolved in 5 minutes; whereas, only 68, 59, 54, and 24% DMF of the two ground, the sieved and the untreated DMF material, respectively, dissolved in 60 minutes.

TABLE 6

| | Particle | | | | | | |
|---|---|---|---|---|---|---|---|
| | Size | % DMF Released | | | | | |
| | Dv50 | 5 | 10 | 20 | 30 | 45 | 60 |
| Samples | (μm) | min | min | min | min | min | min |
| DMF nano-suspen- | 0.123 | 95 | 95 | 96 | 96 | 96 | 96 |
| sion, 200 mg/mL | | | | | | | |
| DMF suspension, | 103 | 51 | 55 | 59 | 61 | 65 | 68 |
| ground 2, 200 | | | | | | | |
| mg/mL | | | | | | | |
| DMF suspension, | 155 | 39 | 42 | 47 | 51 | 54 | 59 |
| ground 1, 200 | | | | | | | |
| mg/mL | | | | | | | |
| DMF suspension, | 183 | 25 | 31 | 38 | 43 | 48 | 54 |
| sieved, 200 mg/mL | | | | | | | |
| DMF suspension, | 453 | 4 | 7 | 10 | 14 | 19 | 24 |
| untreated, 200 | | | | | | | |
| mg/mL | | | | | | | |

All publications, including but not limited to patents and patent applications, cited in this specification are herein incorporated by reference as if each individual publication were specifically and individually indicated to be incorporated by reference herein as though fully set forth.

1. A composition comprising dimethyl fumarate (DMF) nanoparticles.

2. The composition of claim 1, in a form chosen from a liquid form and a solid form.

3. The composition of claim 1, in a form chosen from a powder, a suspension, a hydrogel, an emulsion, a liposome and a micelle.

4. The composition of claim 1, wherein the nanoparticles have a median diameter ranging from about 50 to 400 nm.

5. The composition of claim 1, wherein the nanoparticles have a median diameter ranging from about 100 to 300 nm.

6. The composition of claim 1, wherein the nanoparticles have a median diameter ranging from about 100 to 250 nm.

7. The composition of claim 1, comprising a polymer.

8. The composition of claim 1, comprising a surfactant.

9. The composition of claim 8, wherein the surfactant is an anionic surfactant.

10. The composition of claim 7, wherein the polymer is a cellulose-based polymer.

11. The composition of claim 10, wherein the polymer is chosen from hydroxypropyl cellulose and hydroxypropylmethyl cellulose.

12. The composition of claim 10, wherein the polymer is hydroxypropylmethyl cellulose having a molecular weight in a range of about 2,000 to about 100,000 daltons.

13. The composition of claim 10, wherein the polymer is hydroxypropylmethyl cellulose having a molecular weight in a range of about 10,000 to about 20,000 daltons.

14. The composition of claim 10, wherein the polymer comprises hydroxypropylmethyl cellulose and the nanoparticles have a weight ratio of DMF to hydroxypropylmethyl cellulose in a range of about 1:1 to about 10:1.

15. The composition of claim 10, wherein the polymer comprises hydroxypropylmethyl cellulose and the nanoparticles have a weight ratio of DMF to hydroxypropylmethyl cellulose in a range of about 1:1 to about 6:1.

16. The composition of claim 10, wherein the polymer comprises hydroxypropylmethyl cellulose and the nanoparticles have a weight ratio of DMF to hydroxypropylmethyl cellulose in a range of about 1:1 to about 4:1.

17. The composition of claim 10, wherein the polymer comprises hydroxypropylmethyl cellulose; and the nanoparticles have a weight ratio of DMF to hydroxypropylmethyl cellulose of about 5:1.

18. The composition of claim 9, wherein the anionic surfactant is selected from surfactants having a sulfate, sulfonate, phosphate or carboxylate moiety.

19. The composition of claim 9, wherein the anionic surfactant is dioctyl sodium sulfosuccinate (DOSS).

20. The composition of claim 9, wherein the nanoparticles have a weight ratio of DMF to the anionic surfactant in a range of about 50:1 to about 200:1.

21. The composition of claim 9, wherein the nanoparticles have a weight ratio of DMF to the anionic surfactant in a range of about 50:1 to about 150:1.

22. The composition of claim 9, wherein the nanoparticles have a weight ratio of DMF to the anionic surfactant of about 100:1.

23. The composition of claim 1, comprising DMF, a stabilizer and an anionic surfactant.

24. The composition of claim 1, comprising DMF, hydroxypropylmethyl cellulose and an anionic surfactant.

25. The composition of claim 1, comprising DMF, hydroxypropylmethyl cellulose and dioctyl sodium sulfosuccinate (DOSS).

26. The composition of claim 25, wherein the nanoparticles have a weight ratio of DMF to hydroxypropylmethyl cellulose to DOSS in a range of about 10:2:0.1.

27. A pharmaceutical composition, comprising the composition of claim 1 and a pharmaceutically acceptable carrier.

28. The pharmaceutical composition of claim 27 in a parenteral dosage form comprising a therapeutically effective amount of dimethyl fumarate.

29. The pharmaceutical composition of claim 27 in an oral dosage form comprising a therapeutically effective amount of dimethyl fumarate.

30. The pharmaceutical composition of claim 29, wherein the oral dosage form comprises compressed nanoparticles.

31. The pharmaceutical composition of claim 27 in a form of a tablet, a pill, a capsule, a sustained release formulation, or a powder.

32. A method of treating a disease in a patient in need of such treatment, comprising administering to the patient a therapeutically effective amount of the pharmaceutical composition of claim 27.

33. The method of claim 32, wherein the disease is selected from the group consisting of adrenal leukodystrophy, AGE-induced genome damage, Alexanders Disease, Alper's Disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, diabetic retinopathy, graft versus host disease, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, primary lateral sclerosis, progressive supranuclear palsy, psoriasis, reperfusion injury, retinopathia pigmentosa, Schilders Disease, subacute necrotizing myelopathy, susac syndrome, transplantation rejection, transverse myelitis, a tumor, ulcerative colitis and Zellweger's syndrome.

34. The method of claim 32, wherein the disease is multiple sclerosis.

35. The method of claim 32, wherein the disease is psoriasis.
